(19) United States
(12) Patent Application Publication
(10) Pub. No.: US 2023/0046951 A1
(43) Pub. Date: Feb. 16, 2023

(54) SYSTEM AND METHOD FOR ASSESSING RISK OF TYPE 2 MELLITUS DIABETES COMPLICATIONS

(71) Applicants: National Chengchi University, Taipei City (TW); Kaohsiung Medical University, Kaohsiung City (TW); National Health Research Institutes, Miaoli County (TW)

(72) Inventors: Ming-Yen Lin, Kaohsiung (TW); Jia-Sin Liu, New Taipei City (TW); Ping-Hsun Wu, Kaohsiung (TW); Yi-Wen Chiu, Taipei City (TW); Chih-Cheng Hsu, Miaoli County (TW); Shang-Jyh Hwang, Taipei City (TW); Hsing Luh, New Taipei City (TW)

(73) Assignees: National Chengchi University, Taipei City (TW); Kaohsiung Medical University, Kaohsiung City (TW); National Health Research Institutes, Miaoli County (TW)

(21) Appl. No.: 17/879,796

(22) Filed: Aug. 3, 2022

Related U.S. Application Data
(60) Provisional application No. 63/229,059, filed on Aug. 3, 2021.

(51) Int. Cl. G16H 50/30; G16H 50/20; G16H 10/60

(52) U.S. Cl. CPC G16H 50/30; G16H 50/20; G16H 10/60

(57) ABSTRACT

A system for assessing risks of T2DM complications includes: a data acquisition module obtaining and inputting assessment parameters of a patient with T2DM into a risk assessment module; and the risk assessment module inputting the assessment parameters into a number of risk equations and using it to calculate risk values of the complication occurring after a period of time. The risk equation for all diabetic complications (i,j) is:

rₐ(t,i,j)=1−exp{[H(t₀)−H(t₁)]Cₐ(t,i,j)}

rₐ(t, i, j) is the risk value for the patient to develop the complication j from the current disease i at age t. t₀ is an age of one patient at a state of the disease i. t₁ is an age of the patient after the period of time. t is an age between t₀ and t₁. H(t₀) and H(t₁) are hazards of the complication occurring at the age t₀ and the age t₁, respectively. Cₐ(t, i, j) is a Cox proportional hazards regression expression, and is represented by:

Cₐ(t,i,j)=exp(Rₐ(t,i,j))

Rₐ(t, i, j) is an influence degree of risk factors X on the complication j.

### CROSS-REFERENCE TO RELATED APPLICATION

This application claims the priority benefit of U.S. provisional application Ser. No. 63/229,059, filed on Aug. 3, 2021. The entirety of the above-mentioned patent application is hereby incorporated by reference herein and made a part of this specification.

### TECHNICAL FIELD

The disclosure relates to a risk assessment system and a method for assessing risk. More particularly, the disclosure relates to a system and a method for assessing the risk of type 2 diabetes mellitus complications.

### DESCRIPTION OF RELATED ART

Type 2 diabetes mellitus (T2DM) is a common disease in Taiwan and belongs to the metabolic syndrome. Improper care and management of T2DM may lead directly to permanent disease, including cardiovascular disease, kidney disease, blindness, lower extremity amputations, and death. Especially when patients with T2DM have a disease history of hypertension and/or hyperlipidaemia, the risk of neuropathy and vascular-related complications is significantly increased. Therefore, if the probability of occurrence and occurrence time of T2DM complications are accurately assessed, it helps clinicians and patients to provide better plans to control the development of T2DM.

### SUMMARY

The disclosure provides a system and a method for assessing the risk of T2DM complications, which provide a highly accurate assessment result with meanings in care management.

The system for assessing risks of T2DM complications of the disclosure includes a data acquisition module and a risk assessment module. The data acquisition module obtains assessment parameters of a patient with T2DM and inputs the assessment parameters into the risk assessment module. The risk assessment module inputs the assessment parameters into a number of risk equations and uses the risk equations to calculate risk values of the complications that occur after a period of time. The risk equation is:

rₐ(t,i,j)=1−exp{[H(t₀)−H(t₁)]Cₐ(t,i,j)}

rₐ(t, i, j) is the risk value for one patient to develop a complication j from a current disease i at an age t. t₀ is an age of one patient at a state of the disease i. t₁ is an age of one patient after the period of time. t is an age between t₀ and t₁. H(t₀) and H(t₁) are hazards of the complication occurring at the age t₀ and the age t₁, respectively. Cₐ(t, i, j) is a Cox proportional hazards regression expression, and Cₐ(t, i, j) is represented by the equation below:

Cₐ(t,i,j)=exp(Rₐ(t,i,j))

Rₐ(t, i, j) is an influence degree of multiple risk factors X on the complication.

In an embodiment of the disclosure, the Rₐ(t, i, j) is represented by the equation below:

$${R}_{a} ( t , i , j ) = {β}_{0} + {\sum }_{k} {β}_{k} ( i , j ) {X}_{k} ( a , t )$$

β₀ is an intercept coefficient. βₖ(i, j) is a risk score of a risk factor Xₖ of one patient to an occurrence of the complication j from the disease i in a time interval from the age t₀ to the age t₁. k is a variable from 1 to P, where P is the number of the risk factors X.

In an embodiment of the disclosure, the complication includes end-stage renal disease, arteriosclerotic heart disease, chronic heart failure, ischemic stroke, retinopathy, and amputation.

In an embodiment of the disclosure, the assessment parameters include at least a disease history and the risk factors.

In an embodiment of the disclosure, the disease history includes history of hypertension, history of ischemic stroke, history of arteriosclerotic heart disease, and history of chronic heart failure.

In an embodiment of the disclosure, the risk factors include glycated hemoglobin, systolic blood pressure, body mass index, low-density lipoprotein, high-density lipoprotein, total cholesterol, triglyceride, creatinine, and urine protein and creatinine ratio.

The method for assessing risk of T2DM complications of the disclosure includes: utilizing the system described above to predict the risk value for one patient to develop the complication after the period of time.

Based on the above, in the system and the method for assessing the risk of T2DM complications according to an embodiment of the disclosure, since the risk equation simultaneously considers all risk factors and 62 different disease progressions, the system and the method for assessing the risks of T2DM complications of this embodiment provide a highly accurate assessment result with meanings in care management.

In order to make the above-mentioned features and advantages of the disclosure comprehensible, embodiments accompanied with drawings are described in detail below.

### BRIEF DESCRIPTION OF THE DRAWINGS

FIG. 1 is a tree structure diagram of the disease progression of the T2DM patient.

FIG. 2 shows the assessment parameters of the patients with T2DM in Taiwan's National Health Insurance Research Database.

FIG. 3 shows the absolute error distribution between the actual probability of occurrence and the predicted probability of occurrence.

FIG. 4 shows the absolute percentage error distribution between the actual probability of occurrence and the predicted probability of occurrence.

FIG. 5 is the interface of the “assessment panel” of the system for assessing the risks of T2DM complications of an embodiment of the disclosure.

### DESCRIPTION OF THE EMBODIMENTS

### Embodiment 1: Setting Up a Risk Equation

First, data of patients with T2DM is extracted from Taiwan's National Health Insurance Research Database (NHIRD) referring to the consensus of the experts on the definition. The data includes demographic information diagnoses, laboratory assessment parameters, prescriptions, radiological images, clinical records, etc., but not limited thereto.

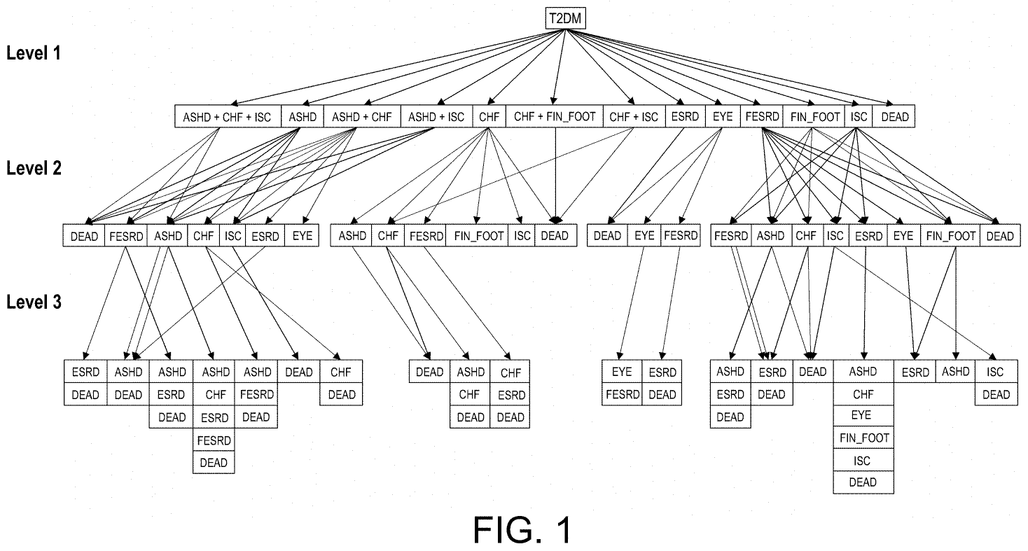

Next, referring to FIG. 1, various possible disease progressions of T2DM patients from the diagnosis to complication occurrence and death (symbol DEAD) are divided into 62 different paths of disease progression, and are presented by a tree structure diagram. In this embodiment, the complications include end-stage renal disease (symbol ESRD), arteriosclerotic heart disease (symbol ASHD), chronic heart failure (symbol CHF), ischemic stroke (symbol ISC), retinopathy (symbol EYE), and amputation (symbol FIN_FOOT).

With continued reference to the tree structure diagram of FIG. 1, the disease progression path of T2DM is subdivided into 2 or 3 levels in the disease development stages. Level 1 in the disease development stages, for example, refers to a path of an occurrence of a first complication in a newly diagnosed T2DM patient after a period of time (e.g., the path of T2DM→ASHD). Level 2 in the disease development stages, for example, refers to a path of a recurrence of the first complication after a period of time in a patient (e.g., the path of T2DM→ASHD→ASHD), a path of an occurrence of a second complication (e.g., the path of T2DM→ASHD→CHF), or a path that leads directly to death (e.g., the path of T2DM→ASHD→DEAD). Level 3 in the disease development stages, for example, refers to a path of a second recurrence of the first complication after a period of time in a patient who once had the first complication recurred (e.g., the path of T2DM→ASHD→ASHD→ASHD), a path of an occurrence of a second complication (e.g., the path of T2DM→CHF→CHF→ASHD), or a path that leads subsequently to death (e.g., the path of T2DM→CHF→CHF→DEAD). Level 3 in the disease development stages may also, for example, refer to a path of a first recurrence of the first complication after a period of time in a patient who had the first complication and the second complication occurred (e.g., the path of T2DM→CHF→FESRD→CHF), a path of an occurrence of a third complication (e.g., the path of T2DM→ISC→CHF→ESRD), or a path that leads subsequently to death (e.g., the path of T2DM→ISC→CHF→DEAD).

With continued reference to the tree structure diagram of FIG. 1, the 62 different paths of disease progression are competitive and independent of each other. For example, when a patient is newly diagnosed with T2DM, the patient's subsequent disease progression (i.e., Level 1 in the disease development stages) has the following possibilities: ASHD+CHF+ISC, ASHD, ASHD+CHF, ASHD+ISC, CHF, CHF+, CHF+ISC, ESRD, EYE, FESRD, FIN_FOOT, ISC, and DEAD. When a patient develops a path of T2DM→EYE in Level 1 in the disease development stages, the patient's subsequent disease progression (i.e., Level 2 in the disease development stages) has a greater probability of developing the paths of DEAD, EYE, or FESRD, but only a small probability of developing the paths of ASHD, CHF, ISC, and FIN_FOOT. That is, a patient's current disease progression (including complication status) has a great influence on the subsequent disease development.

Next, the data of the patients with T2DM is screened, and the assessment parameters usable for simulation are regarded as the initial value. After that, computer simulation technique is used to develop the risk equations for simulating the 62 different disease progressions, so that the risk equations are capable of considering the multi-level disease progressions (or path) and are subsequently used to accurately assess the risks of complications in the patients with T2DM after a period of time. That is, the risk equations should be capable of assessing the risks of complications in the patients after a period of time based on the patient's current disease progression (including complication status).

In the process of developing risk equations for this embodiment, the assessment parameters of the patients with T2DM include at least age, gender, disease history (e.g., history of hypertension, history of ischemic stroke, history of arteriosclerotic heart disease, and history of chronic heart failure), glycated hemoglobin (HbA1c), systolic blood pressure (SBP), body mass index (BMI), low-density lipoprotein (LDL), high-density lipoprotein (HDL), total cholesterol (TC), triglyceride (TG), creatinine (Cr), and urine protein and creatinine ratio (UPCR), but not limited thereto. In this embodiment, the assessment parameters of the patients with T2DM are obtained, for example, by non-invasive inspections (e.g., general physiological data and urinalysis) and standard health examination (e.g., vitro experimental testing related to blood samples obtained from blood drawing).

In this embodiment, the computer simulation techniques include statistical methods such as nonhomogeneous Markov chain, Cox proportion hazards ratio model, regression learning algorithm of Bayesian approach, and Weibull distribution, etc., but not limited thereto.

In this embodiment, a number of risk equations are used to evaluate: the risk of a first complication after a period of time in patients newly diagnosed with T2DM; the risk of recurrence of the complication after a period of time in patients with T2DM who had previously had complication, the risk of another complication after a period of time in patients with T2DM who had previously had complication, but not limited thereto. In addition, the risk equation also evaluates the probability and sequence of which complication or death is going to occur next based on the patient's current stage in disease development (including complication status). The complications of patients with T2DM appears according to the time points of the Weibull distribution.

In this embodiment, the risk equation describes the patient's status change with the nonhomogeneous Markov chain and expresses the influence of the initial value on status transitions with the Cox proportion hazards ratio model. The risk equation of this embodiment for all diabetic complications (i,j) is:

rₐ(t,i,j)=1−exp{[H(t₀)−H(t₁)]Cₐ(t,i,j)}.

In the risk equation, rₐ(t, i, j) is a risk value for one patient to occur the complication j to be assessed from the current disease progression i when the patient reaches age t. t₀ is the age of the patient. t₁ is the age of the patient after a period of time. t is the age between t₀ and t₁. i is the current disease progression. j is the complication to be assessed. H(t₀) is the hazard of the complication to be assessed occurring at age t₀ in the Weibull distribution. H(t₁) is the hazard of the complication to be assessed occurring at age t₁ in the Weibull distribution. Cₐ(t, i, j) is a function of the Cox proportional hazards regression expression.

The risk value rₐ(t, i, j) may represent the probability of occurrence of the complication j and the sequence thereof. For example, if the risk value of complication A after assessment is 1% and the risk value of complication B after assessment is 0.5%, the probability of occurrence of complication A is greater than that of complication B, and complication A occurs before complication B.

In the risk equation, the function of the Cox proportional hazards regression expression Cₐ(t, i, j) is represented by the equation below:

Cₐ(t,i,j)=exp(Rₐ(t,i,j)).

In the function of the Cox proportional hazards regression expression, Rₐ(t, i, j) is an influence degree of the risk factors X on the complication j to be assessed. Rₐ(t, i, j) is represented by the linear regression equation below:

$${R}_{a} ( t , i , j ) = {β}_{0} + {\sum }_{k} {β}_{k} ( i , j ) {X}_{k} ( a , t ) .$$

In the linear regression equation of Rₐ(t, i, j), β₀ is an intercept coefficient of the linear regression equation. βₖ(i, j) is a slope of the linear regression equation. βₖ(i, j) is a risk score of a risk factor Xₖ of a patient to the occurrence of the complication j to be assessed from the disease i in the time interval from the age t₀ to the age t₁. k is a variable from 1 to P, where P is a number of the risk factors X. Therefore, the linear regression equation of Rₐ(t, i, j) is used to simultaneously consider the influence degree of all risk factors X on the complication to be assessed.

In the linear regression equation of Rₐ(t, i, j), βₖ(i, j) is obtained by the regression learning algorithm of the Bayesian approach.

In the risk equation, H(t₀) and H(t₁) are represented by the equation below:

h(t|X(t))=h₀(t)exp(X(t)*β).

X(t) is the patient risk factor at age t. β is the correlation coefficient. h₀(t) is the risk ratio of the occurrence of the complications to be assessed at age t in the Weibull distribution. h₀(t) is presented by the equation below:

h₀(t)=λκt^{κ-1}.

In the equation of h₀(t), κ is the shape parameter, λ is the scale parameter, and λ=exp(β₀).

### Embodiment 2: Establishing a System for Assessing the Risks of Type 2 Diabetes Mellitus Complications

In this embodiment, a risk assessment system for T2DM complications is established as a user-friendly visual support decision-making system. Therefore, health care workers such as doctors, nurses, or health care providers may predict the risks of complications in the future as a reference for disease control and management by inputting the assessment parameters of the patients.

Specifically, the risk assessment system for T2DM complications of this embodiment includes a data acquisition module and a risk assessment module. The data acquisition module is used to obtain assessment parameters of a patient with T2DM and input the assessment parameters into the risk assessment module. The risk assessment module is used to input the assessment parameters into the risk equations and uses the risk equations to calculate risk values or probabilities of occurrence of various complications after a period of time.

In this embodiment, the assessment parameters may include the age of the patient, the disease history related to the complication, and risk factors related to the complications. The disease history related to the complications may include history of hypertension, history of ischemic stroke, history of arteriosclerotic heart disease, and history of chronic heart failure, but not limited thereto. The risk factors related to the complications may include glycated hemoglobin, systolic blood pressure, body mass index, low-density lipoprotein, high-density lipoprotein, total cholesterol, triglyceride, creatinine, and urine protein and creatinine ratio, but not limited thereto.

In this embodiment, the data acquisition module may obtain the assessment parameters of one patient with T2DM by the input of the assessment parameters of the patient by the health care workers such as doctors, nurses, or health care providers to the system for assessing the risks.

In this embodiment, after the assessment parameters are inputted into the risk assessment module by the data acquisition module and before the risk values (or the probabilities of occurrence) of suffering from various complications after a period of time is calculated by the risk assessment module using a number of risk equations, it is also necessary to select the “risk assessment pattern” to be used and the “disease progression path” to be assessed.

In this embodiment, the “risk assessment pattern” includes two patterns, which are “absolute value basis” and “relative value basis”. The “absolute value basis” refers to the probability of occurrence of complications presented as a percentage. That is, how many people will suffer from this complication per 100 people. However, the incidences of most complications are low due to the large number of patients. The “relative value basis” is based on taking the probability of occurrence of one of the complications as a benchmark, the probability of occurrence of other complications are multiples of the probability of occurrence of the one of the complications. Taking, for example, the probability of occurrence of ASHD as the benchmark, the probability of occurrence of other complications are multiples of the probability of occurrence of ASHD.

In this embodiment, the “disease progression path” may include “a stage from newly diagnosed with T2DM to the occurrence of the first complication” (e.g., Level 1 in the disease development stages in the tree structure diagram of FIG. 1) and “a stage from the occurrence of the first complication to the occurrence of a second complication” (e.g., Level 2 and Level 3 in the disease development stages in the tree structure diagram of FIG. 1). Therefore, patients should choose the stage in disease development to be assessed based on their own current disease progression.

In this embodiment, after the risk values (or the probabilities of occurrence) of suffering from various complications after a period of time is calculated by the risk assessment module using a number of risk equations, the system outputs the prediction result of the risk values (or the probabilities of occurrence) into a prediction report and provide to one patient. The patient may manage the disease based on the prediction report, thereby reducing the occurrence of complications and improving the quality of life of the patient.

In some embodiment, after the risk values (or the probabilities of occurrence) of suffering from various complications after a period of time are calculated by the risk assessment module using a number of risk equations, the various possible complications may be sorted according to the risk values (or the probabilities of occurrence), so that health care workers and patients may make care decisions according to the sorted results and improve care quality.

In addition, the inventors emphasized that since the disease progression of T2DM patients in Asia is different from that in Western countries (e.g., ASHD, ESRD, and ISC are common complications in T2DM patients in Asia, but not CHF, ASHD, EYE, and FIN_FOOT). Therefore, when compared to the risk assessment system for T2DM patients in Western countries, the risk assessment system of this embodiment is more suitable for type 2 diabetic patients in Asia.

### EXPERIMENTAL EXAMPLE

### <Experimental Example 1> Verifying the Accuracy of the Risk Equation

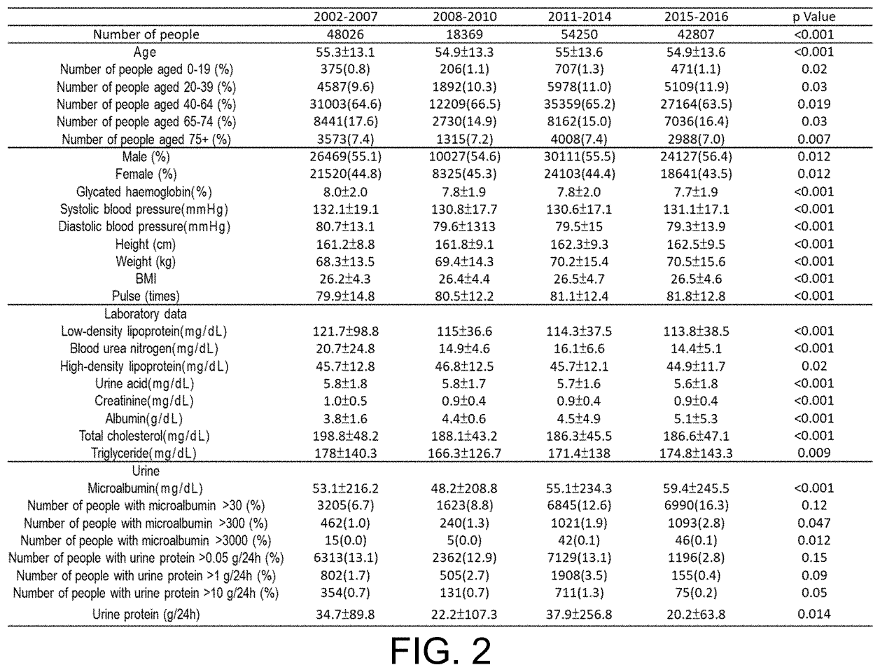

First, the baseline of the assessment parameters of a total of 163,452 T2DM patients was extracted from a subset of the Taiwan's National Health Insurance Research Database at different time intervals (2002-2007, 2008-2010, 2011-2014, 2015-2016), as shown in FIG. 2. Where the age was 54.00±11.86 years old, the proportion of female was 44.40%, the history of diabetes was 5.56±6.28 years, the glycated hemoglobin was 7.8±2.10%, the body mass index was 26.481±3.97 kg/m², the triglyceride was 172.64±135.51 mg/dL, the low-density lipoprotein was 116.13±35.28 mg/dL, the systolic blood pressure was 130.65±18.16 mmHg, and the diastolic blood pressure was 79.77±13.82 mmHg. Next, the T2DM patients' disease progression and assessment parameters were continuously tracked over a long period of time (about 16 years or more). Then, before carrying out the risk assessment, we first excluded 20,835 patients without a history of complications and risk factors. Then, 116,692 patients were excluded due to fewer than 10 years of observational time. Next, 13,683 patients who participated in P4P project for the first time between 2002-2005 were also excluded. Finally, the risk assessment was performed on 12,242 patients with at least one complication.

Next, the 12,242 patients were assessed for the risk of complications by using a number of risk equations. Then, the risk values (probabilities of occurrence) obtained after the risk assessment were compared with the actual value of the actual complications of these patients, and the comparison result is as follows:

1. Taking the risk assessment result of ASHD as an example, the risk assessment result of ASHD indicates that the probability of occurrence of ASHD in T2DM patients aged 55 to 60 increased with age, for example, from 0.05 to 0.1; the actual probability of occurrence of ASHD in T2DM patients aged 55 to 60 years was almost the same as the predicted probability of occurrence (or risk value); the actual probability of occurrence of ASHD in T2DM patients aged 57 to 58 was slightly lower than the predicted probability of occurrence (or risk value), but still within the predicted interval; the actual probability of occurrence of ASHD in patients with T2DM aged 59 to 60 years was higher than the predicted probability of occurrence (or risk value).

2-1. Among the 12,242 patients, the actual number and annual rate of the first complication of ASHD within 14.5 years were 2015 and 0.0114, respectively; and the predicted number and annual rate of the first complication of ASHD within 14.5 years were 2268 and 0.0128, respectively.

2-2. Among the 12,242 patients, the actual number and cumulative probability of occurrence of recurrence of ASHD within 14.5 years were 284 and 0.0016; and the predicted number and cumulative probability of occurrence of recurrence of ASHD within 14.5 years were 309 and 0.0017.

2-3. Among the 12,242 patients, the actual number and cumulative probability of occurrence of the first complication of ISC within 14.5 years were 549 and 0.0031; and the predicted number and cumulative probability of occurrence of the first complication of ISC within 14.5 years were 594 and 0.0033, respectively.

2-4. Among the 12,242 patients, the actual number and cumulative probability of occurrence of recurrence of ISC within 14.5 years were 22 and 0.0001; and the predicted number and cumulative probability of occurrence of recurrence of ISC within 14.5 years were 29 and 0.0002.

2-5. Among the 12,242 patients, the actual number and cumulative probability of occurrence of the first complication of CHF within 14.5 years were 828 and 0.0047; and the predicted number and cumulative probability of occurrence of the first complication of CHF within 14.5 years were 780 and 0.0044, respectively.

2-6. Among the 12,242 patients, the actual number and cumulative probability of occurrence of the first complication of ESRD within 14.5 years were 2,250 and 0.0127; and the predicted number and cumulative probability of occurrence of the first complication of ESRD within 14.5 years were 2,268 and 0.0128, respectively.

3. The “actual probability of occurrence of the first complication and death of 12,242 patients within 10 years” and the “predicted probability of occurrence (or risk value) of the first complication and death of 10,000 simulated patients” are recorded in Table 1. The characteristics of the 10,000 simulated patients are roughly the same as the characteristics of the 12,242 patients, and the predicted probability of occurrence (or risk value) in Table 1 is the average value after 50 simulations and calculations.

TABLE 1

| | actual probability | predicted probability |
|---|---|---|
| | of occurrence | of occurrence |
| DEAD | 11.0% | 12.3% |
| ASHD | 1.9% | 4.5% |
| EYE | 0.0% | 3.2% |
| ASHD + CHF | 0.6% | 3.5% |
| ESRD | 3.1% | 2.8% |
| ISC | 0.6% | 2.6% |
| CHF | 0.4% | 2.2% |
| FIN_FOOT | 0.1% | 1.8% |
| CHF + ISC | 0.0% | 0.0% |
| CHF + FIN_FOOT | 0.0% | 0.1% |
| ASHD + ISC | 0.1% | 0.3% |

According to the results in Table 1, the difference between the actual probability of occurrence and the predicted probability of occurrence of ESRD is the smallest (about 0.3%), while the difference between the actual probability of occurrence and the predicted probability of occurrence of EYE is the greatest (about 3.2%). The difference between the actual probability of occurrence and the predicted probability of occurrence of ASHD is 2.60%, the difference between the actual probability of occurrence and the predicted probability of occurrence of ASHD+CHF is 2.90%, the difference between the actual probability of occurrence and the predicted probability of occurrence of ISC is 2.00%, the difference between the actual probability of occurrence and the predicted probability of occurrence of CHF is 1.80%, the difference between the actual probability of occurrence and the predicted probability of occurrence of FIN_FOOT is 1.70%, the difference between the actual probability of occurrence and the predicted probability of occurrence of CHF+ISC is 0.00%, the difference between the actual probability of occurrence and the predicted probability of occurrence of CHF+FIN_FOOT is 0.10%, and the difference between the actual probability of occurrence and the predicted probability of occurrence of ASHD+ISC is 0.20%. In addition, the difference between the actual probability of occurrence and the predicted probability of occurrence of death is 1.30%.

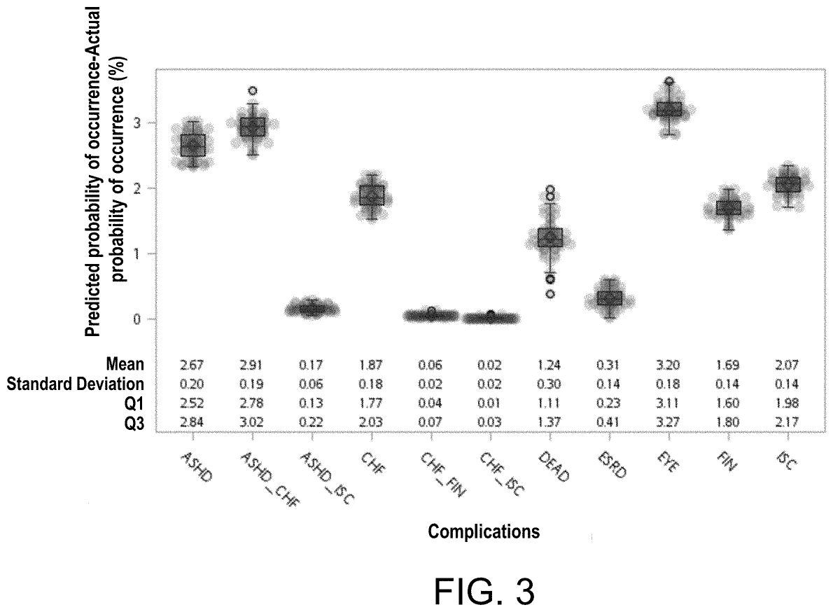

4. The absolute errors between the “actual probability of occurrence of the first complication and death of 12,242 patients within 10 years” and the “predicted probability of occurrence (or risk value) of 10,000 simulated patients calculated using the risk equation” are illustrated in FIG. 3. The characteristics of the 10,000 simulated patients are roughly the same as the characteristics of the 12,242 patients, and the circle distribution of FIG. 3 represents the difference between the predicted probability of occurrence (or risk value) and the actual probability of occurrence after 50 simulations and calculations.

According to the result in FIG. 3, the absolute error between the actual probability of occurrence and the predicted probability of occurrence (or risk value) of the first complication (or death) is within 5%.

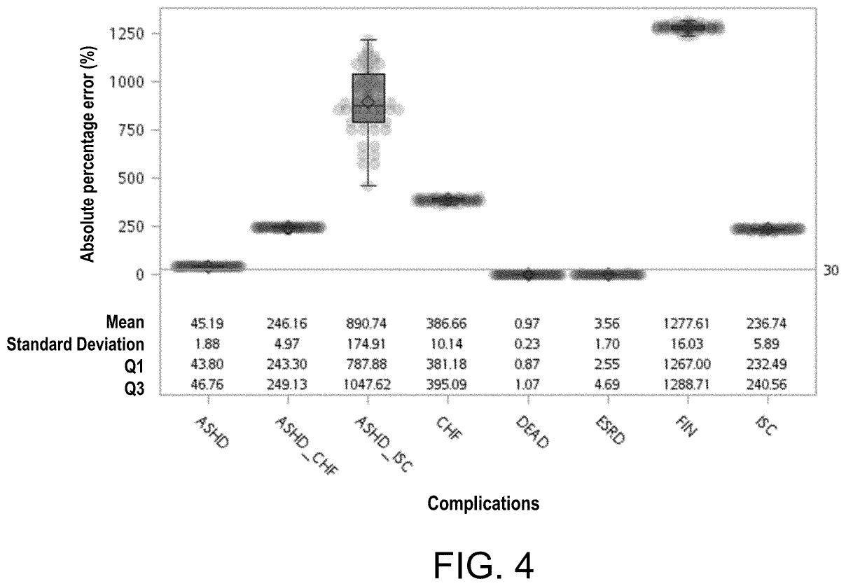

5. The absolute percentage errors between the “actual probability of occurrence of the first complication and death of 12,242 patients within 10 years” and the “predicted probability of occurrence (or risk value) of 10,000 simulated patients calculated using the risk equation” are illustrated in FIG. 4. The characteristics of the 10,000 simulated patients are roughly the same as the characteristics of the 12,242 patients, and the circle distribution in FIG. 4 represents the difference between the predicted probability of occurrence (or risk value) and the actual probability of occurrence after 50 simulations and calculations.

According to the result of FIG. 4, the absolute percentage errors of ASHD, death, and ESRD are all within the generally acceptable range of 30%. The probability of occurrence of ISC and CHF have moderate errors in the model estimation, and the predicted probability of occurrence of FIN_FOOT is overestimated.

Based on the above, since the risk equation of this embodiment produces a similar predicted probability of occurrence (or risk value) with the actual probability of occurrence for T2DM complication patients, the risk assessment system for T2DM complication of this embodiment may provide a highly accurate assessment result with meanings in care management.

### <Experimental Example 2> Application of the System for Assessing the Risks of Type 2 Diabetes Mellitus Complications

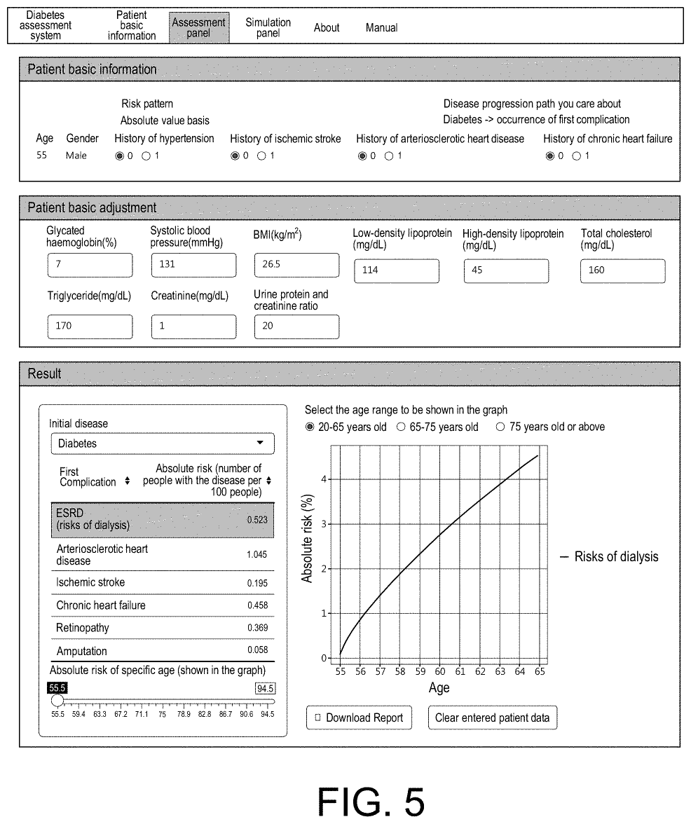

Referring to FIG. 5, in the risk assessment system for T2DM complications, the user first input the age as 55 years old, the gender as male, no history of hypertension (code 0), no history of ischemic stroke (code 0), no history of arteriosclerotic heart disease (code 0), and no history of chronic heart failure (code 0) in the “patient basic information” interface. Next, the “absolute value basis” and “a stage from newly diagnosed with T2DM to the occurrence of the first complication” were selected according to the needs of the patient as the “risk assessment pattern” and the “disease progression path” respectively.

Then, in the “assessment panel” interface, in addition to the previously entered “patient basic information”, the user continued inputting the glycated hemoglobin as 7%, the systolic blood pressure as 131 mmHg, the body mass index as 26.5 kg/m², the low-density lipoprotein as 114 mg/dL, the high-density lipoprotein as 45 mg/dL, the total cholesterol as 160 mg/dL, the triglyceride as 170 mg/dL, the creatinine as 1 mg/dL, and the urine protein and creatinine ratio as 20 in the “patient information adjustment” column.

A table (left side), a predictable specific age range (lower left side), and a graph (right side) were then provided in the “result presentation” column. The predictable specific age range of the risk assessment system was +0.5 years old to +39.5 years old of the patient's age. The risk value (or probability of the occurrence) of various complications for a specific age was displayed in the table. The trend of the risk value (or probability of the occurrence) of specific complication for a selected age range (including 20 to 65 years old, 65 to 75 years old, and 75 years old and above) was displayed in the graph. That is to say, if other specific ages are selected, the risk value (or probability of occurrence) displayed in the table and the trend of the risk value (or probability of occurrence) displayed in the graph also change accordingly.

Referring to FIG. 5, FIG. 5 shows that the predictable specific age range was 55+0.5 years old to 55+39.5 years old, and the currently selected specific age was 55.5 years old. In the table, the risk value of ESRD was 0.523%, the risk value of arteriosclerotic heart disease was 1.045%, the risk value of ischemic stroke was 0.195%, the risk value of chronic heart failure was 0.458%, the risk value of retinopathy was 0.369%, and the risk value of amputation was 0.058%. The graph shows that after 10 years (when the patient is about 65 years old), the risk value (or probability of occurrence) of ESRD will be increased from 0.523% to about 4.5%.

To sum up, in the system and the method for assessing the risks of T2DM complications according to an embodiment of the disclosure, since the risk equation simultaneously considers all risk factors and 62 different disease progressions, the system and the method for assessing the risks of T2DM complications of this embodiment provide a highly accurate assessment result with meanings in care management.

Although the disclosure has been described in detail with reference to the above embodiments, they are not intended to limit the disclosure. Those skilled in the art should understand that it is possible to make changes and modifications without departing from the spirit and scope of the disclosure. Therefore, the protection scope of the disclosure shall be defined by the following claims.

What is claimed is:

1. A system for assessing risks of T2DM complications, comprising:
a data acquisition module, obtaining assessment parameters of a patient with T2DM and inputting the assessment parameters into a risk assessment module; and
the risk assessment module, inputting the assessment parameters into a number of risk equations and using the risk equations to calculate risk values of the complications that occur after a period of time,
wherein the risk equation is:
rₐ(t,i,j)=1−exp{[H(t₀)−H(t₁)]Cₐ(t,i,j)}
wherein rₐ(t, i, j) is the risk value for the patient to develop a complication j from a current disease i at an age t,
t₀ is an age of the patient at a state of the disease i,
t₁ is an age of the patient after the period of time,
t is an age between t₀ and t₁,
H(t₀) and H(t₁) are hazards of the complication occurring at the age t₀ and the age t₁, respectively, and
Cₐ(t, i, j) is a Cox proportional hazards regression expression, represented by:
Cₐ(t,i,j)=exp(Rₐ(t,i,j))
wherein Rₐ(t, i, j) is an influence degree of a plurality of risk factors X on the complication j.

2. The system according to claim 1, wherein the Rₐ(t, i, j) is represented by:
$${R}_{a} ( t , i , j ) = {β}_{0} + {\sum }_{k} {β}_{k} ( i , j ) {X}_{k} ( a , t )$$
wherein β₀ is an intercept coefficient,
βₖ(i, j) is a risk score of a risk factor Xₖ of the patient to an occurrence of the complication j from the disease i in a time interval from the age t₀ to the age t₁, and
k is a variable from 1 to P, wherein P is the number of the risk factors X.

3. The system according to claim 1, wherein the complication comprises end-stage renal disease, arteriosclerotic heart disease, chronic heart failure, ischemic stroke, retinopathy, and amputation.

4. The system according to claim 1, wherein the assessment parameters comprise at least a disease history and the risk factors.

5. The system according to claim 4, wherein the disease history comprises history of hypertension, history of ischemic stroke, history of arteriosclerotic heart disease, and history of chronic heart failure.

6. The system according to claim 1, wherein the risk factors comprise glycated hemoglobin, systolic blood pressure, body mass index, low-density lipoprotein, high-density lipoprotein, total cholesterol, triglyceride, creatinine, and urine protein and creatinine ratio.

7. A method for assessing risks of T2DM complications, comprising:
utilizing the system according to claim 1 to predict the risk value for the patient to develop the complication after the period of time.
